(19) United States
(12) Patent Application Publication
(10) Pub. No.: US 2013/0263875 A1
(43) Pub. Date: Oct. 10, 2013

(54) CURABLE NAIL ENHANCEMENTS

(71) Applicant: ESSCHEM, INC., Linwood, PA (US)

(72) Inventors: Tamara Burgess, Clementon, NJ (US); Angeline B. Louis, Hatboro, PA (US)

(73) Assignee: Esschem, Inc., Linwood, PA (US)

(21) Appl. No.: 13/852,642

(22) Filed: Mar. 28, 2013

Related U.S. Application Data
(60) Provisional application No. 61/749,970, filed on Jan. 8, 2013.
(60) Provisional application No. 61/710,923, filed on Oct. 8, 2012.
(60) Provisional application No. 61/622,043, filed on Apr. 10, 2012.

(51) Int. Cl. A45D 29/00; A61Q 3/02; A61K 8/72

(52) U.S. Cl. CPC A45D 29/00; A61K 8/72; A61Q 3/02
USPC 132/200; 424/61; 132/73

(57) ABSTRACT

Monophasic energy-curable solvent-free compositions useful for providing decorative nail enhancement are formulated using at least one energy-curable resin and at least one film-former such as a polyester or siloxane. Good adhesion to the nail surface is achieved without the use of a base coat and a top coat is not needed in order to attain an outer surface having acceptable esthetic appearance. The cured composition exhibits good durability and yet can be readily and cleanly removed when desired by soaking briefly in acetone.

### CROSS-REFERENCE TO RELATED APPLICATIONS

This application claims priority to U.S. Provisional Application Nos. 61/749,970, filed 8 Jan. 2013; 61/710,923, filed 8 Oct. 2012; and 61/622,043, filed 10 Apr. 2012, each of which is incorporated herein by reference in its entirety for all purposes.

### FIELD OF THE INVENTION

The invention relates to curable liquid or gel products useful for the decorative enhancement of mammalian nails.

### BACKGROUND OF THE RELATED ART

Nail enhancement is thought to date back to the Incas, who decorated their fingernails with different tints, paints, and even pictures of animals. By the turn of the 19^{th} century, simple painting of the nail was not as desirable as having polished nails with a glossy varnish finish. This was achieved by tinting the nails with scented red oils and then buffing with chamois cloth to achieve the final polished and shiny appearance. In 1920, high gloss lacquer automobile paint was created, which inspired the introduction of colored nail polish.

Other than its fashionable attribute, nail polish also provided protection to the fingernail to prevent nail splitting, contact with detergents, and evaporation of water from the nail plate. Nail polish initially lacked functional attributes such as easy application, rapid drying and hardening, water resistance, adhesion, elasticity, chipping resistance and high gloss. As nail polish technology evolved, film-forming agents, resins, plasticizers, and solvents were incorporated to provide these attributes.

Currently, there is market demand for extended wear and durability to couple with the advantages already known of nail cosmetics. Most nail enhancements provide a coating over the nail plate to hide flaws. Many persons perceive painted nails as more attractive and so, wear artificial nails for a longer time.

Nail polish is still one of two commonly used coatings for nails. Nail polish hardens upon solvent evaporation, while the second type of coating polymerizes. Many types of products are used to create this second group of polymerized coatings, or artificial nail enhancements. Acrylics are utilized in one of three primary enhancement systems: powder/liquid sculptured nails (conventionally known as “acrylic nails”), silk wraps, and ultraviolet (UV) polymerized acrylic gels. The end result of each is a smooth, attractive, blemish-free nail enhancement.

Acrylic nails are used to artificially enhance the appearance of natural fingernails. Acrylic resin was first employed with powder/liquid systems, in which a powder and liquid are mixed to form a thick paste. After preparing the nail with a primer (such as methacrylic acid or a non-acidic primer), which improves bonding to the real nail, the salon technician applies or “sculpts” the paste into place over the natural nail and allows it to polymerize. Acrylic nails cure with a free radical reaction, initiated when the peroxide in powder is exposed to the reactive monomer in the liquid. When the resin hardens, it is filed into the desired shape. Today, acrylic chemistry is used to make a variety of nail enhancements, including nail tips, overlays, wraps, and sculpted nails.

Due to their lack of odor, gels have become popular in the beauty salon industry. Gel nail enhancements are thinner and less durable than acrylic nails, but provide a smooth surface. Gel nails are generally composed of acrylates, but with different molecular structures, appearance, application procedures and curing methods. These gels contain acrylates, methacrylate and urethane compounds, colorants and a photoinitator. The gel remains in a semi-liquid form until cured in a photo-bonding box containing a UV and/or LED (light emitting diode) light source. When the gel is exposed to the light of an appropriate wavelength, polymerization occurs, resulting in hardening of the gel. Gel nails have been marketed with the promises of: easier application, faster drying, longer life, and easier removal, with less weakening of the natural nail plate. Even with fulfillment of these claims, improvements to nail enhancements are still desired. These further improvements include making the nail enhancement strong yet flexible, hard but not brittle, and creating a natural appearing nail.

Nail gel viscosities broadly range from 10,000 to 120,000 cPs at room temperature. Such thick mixtures, which can have a consistency between that of molasses and petrolatum, require that the gel be dispensed from shallow open head containers (called pots) and applied to the nail using a fine bristled gel brush.

Cured nail enhancements present removal challenges. Acrylics are usually removed with a mechanical grinder. Even with professional use, the high speed rotation of the grinder head will come in contact with some part of and may damage the natural nail. In early iterations, gels were also removed with a mechanical grinder. Over time, products began to incorporate more methacrylate species, which allows for slow solvent attack, in that the gel softens and can be gently scraped off the natural nail. Removal was thereby improved to soaking fingertips in acetone for a considerable length of time. Gels which can be removed with acetone are known as “soak off” gels.

By 2011, a number of published patent applications had described UV curable brush-on gels. Brush-on gels are lower in viscosity (less than 10,000 cPs), with application similar to nail polish, hence the term “gel polish” or “gel lacquer”. Typically, a gel polish component will have a viscosity and/or working attributes comparable to traditional nail lacquer. Predominant gel polish systems are comprised of three components: a base-coat, a color layer, and a top-coat. Each part of the system is typically applied from a nail polish bottle, the presentation of which creates consumer recognition.

Similar to historical primers, such as are used in powder-liquid systems, the principle function of the base coat or bonding layer is to provide adhesion to the natural nail. This base coat serves as an interface with the natural nail and creates a favorable surface for application of additional coating layers. After curing the base coat, and each subsequent coat, the color layer is applied over the base coat. Usually, a second coat of color is applied, for better opacity and enhanced durability. A third, final layer is applied on top of the color layer. This layer, which is called the top coat, gloss-coat, finish coat or protective layer, provides shine to the nail. The development of gel polishes which can be applied as a monophasic colored coat (i.e., without the need for either a base coat or a top coat) and yet still provide the needed nail adhesion, durability, and esthetic appearance would be advantageous.

Gel polishes often incorporate the same types of solvents as nail polish. The solvent(s) aid with applying the gel as a smooth coating on the nail. When the solvents evaporate, per the hypothesis of certain patent applications [US 2011/0060065, US 2011/0182838 and/or US 2011/0274633], remaining are a network of pores or channels within the coating matrix. At the time of solvent removal, the additional internal surface area allows the gel to soak-off faster than soak-off gels. After soaking the nails in solvent, soak-off gels are known to remove in 10-30 minutes. Gel polishes known in the industry remove in 10-20 minutes, comparatively. It would be desirable to develop nail polishes that can be easily applied to the nail surface to provide a smooth coating and that can be quickly removed after curing by soaking in solvent, yet do not rely on the use of solvents in their formulation.

It is also known that the solvent in both nail and gel polish evaporate when the bottle cap is removed. With both, the evaporation leads to an imbalance of the recipe, subsequent solidification in the bottle and product loss. It would be desirable to develop nail polishes from which a greater yield per bottle can be realized.

Because they are so fluid, gel polishes may exhibit “pull-back,” which is a reduction in the area coated by wet polish, most frequently seen at the edges of the nail. During application of a curable gel polish, the natural surface tension and irregular surface topography of a nail causes the uncured polish to pull-back (or shrink away) from the cuticle, side wall or free edge of the nail. It may be assumed this effect is demonstrating poor interfacial compatibility between the liquid polish and solid nail, and that the wet polish has a high contact angle. The concentrated wet polish then puddles, pools and/or runs off the nail. Workers in the field have recognized that an ideal nail polish would exhibit little or no such pull-back when applied to nails.

### BRIEF SUMMARY OF THE INVENTION

A composition useful as a photocurable nail gel or liquid which requires no base coat or top coat when applied to a nail is provided by the present invention. Because the composition need not be applied in combination with a base coat or top coat in order to attain good adhesion to the nail and good appearance and durability, it may be considered “monophasic” (wherein the finished, enhanced nail only bears one or more layers of the cured composition). The composition comprises at least one film-former (such as a polyester or siloxane) and at least one energy-curable resin and contains essentially no water or volatile non-reactive organic solvent.

A composition in accordance with the invention may possess one or more of the following desirable characteristics:
1) Will not pull away from the cuticle, side wall or free edge of the nail during application;
2) Has a high gloss shine after UV and/or LED cure;
3) Has good adhesion to the nail for at least 10 or at least 14 days;
4) Will not damage the nail when soaked off using acetone or when peeled off by hand;
5) Will not discolor the nail bed after removal; and
6) Will require less than 10 minutes of soak time in acetone to remove from the nail.

### DETAILED DESCRIPTION OF CERTAIN EMBODIMENTS OF THE INVENTION

The terms “nail” and “nail surface” as used herein mean the natural, keratinaceous nail surface. The compositions of the invention may be applied directly to the keratinaceous surface of a natural nail (e.g., fingernail, toenail) of a mammal such as a human being. One significant advantage of the present invention is that pretreatment of the nail surface is not needed in order to achieve good adhesion of the cured composition. Thus, a base coat or primer need not be applied before placing a layer of the composition in accordance with the invention onto the nail.

The inventive compositions described herein are polymerizable (curable) liquids or gels which are capable, when applied to a nail surface, of forming a smooth, relatively even layer which completely conforms to the nail surface. The compositions may be cured using radiant energy, in particular actinic radiation (electromagnetic radiation that can produce photochemical reactions) such as ultraviolet (UV) or visible light radiation, such as LED.

The compositions of the present invention contain at least one film-former and at least one energy-curable (e.g., photocurable) resin. Additional components may also be present such as, for example, one or more colorants (e.g., dyes, pigments), photoinitiators, fillers, rheology modifiers, thixotropic agents, plasticizers, UV absorbers, UV stabilizing agents, dispersants, and the like. The compositions are characterized, however, by the absence or substantial absence of water as well as volatile non-reactive organic solvents (e.g., less than 0.1 weight % total of such substances). As used herein, the term “volatile” means a compound having a boiling point at atmospheric pressure of 200° C. or less.

Film formers are well established in industry. Makers of cosmetics and personal care products have used film formers in many recipes and in particular, conventional nail polish.

The film-former component of the curable compositions of the present invention has been found to be critical with respect to attaining certain desired properties, in particular the ability to create a film coating which self-levels without the use of non-reactive solvents. In the classic sense of a surfactant reducing the solid/liquid interfacial tension, the use of a film-former in the composition, particularly polyester and/or siloxane film-formers, enables the composition to wet the natural nail surface, thereby reducing the contact angle of the uncured liquid. By neutralizing this interface, the polyester or siloxane film-former serves to alleviate the “pull-back” phenomena previously experienced with solvent-free nail gel polish compositions, and assists in providing a finished nail gel polish film which is smooth, pliable and continuous. Suitable film-formers include oligomeric and especially polymeric substances, which can be either reactive or non-reactive (i.e., not capable of being cured using energy such as UV light) as well as non-volatile and which are capable of forming films.

Suitable film-formers include, in particular, polyesters. Preferred are those polyesters such as obtained by condensation of aromatic and aliphatic di- and tri-carboxylic acids (such as, phthalic acid, isophthalic acid, terephthalic acid, adipic acid, azelaic acid, glutaric acid, maleic acid, fumaric acid, dimeric fatty acids, sebacic acid, itaconic acid, trimellitic acid, pyromellitic acid and the like), and anhydrides thereof, with di- and tri-alcohols (such as glycols, including ethylene glycol, propylene glycol, neopentyl glycol, glycerol, trimethylene glycol, tetraethylene glycol, triethylene glycol, dipropylene glycol, trimethylolpropane, diethylene glycol, pentaerythritol and the like).

Other types of film-formers useful in the practice of the invention include acrylates and acrylate copolymers, polyamides, polyolefins and olefin copolymers such as olefin/maleic anhydride copolymers, PVP copolymers, styrene copolymers, vinyl acetate copolymers, and the like. Such film formers may include, but are not limited to, acrylamide/sodium acrylate copolymer, acrylamides copolymer, acrylamides/acrylates/DMAPA/methoxy PEG methacrylate copolymer, acrylamidopropyltrimonium chloride/acrylates copolymer, acrylates copolymer, acrylates/acetoacetoxyethyl methacrylate copolymer, acrylates/acrylamide copolymer, acrylates/ammonium methacrylate copolymer, acrylates/carbamate copolymer, acrylates/ceteth-20 itaconate copolymer, acrylates/ceteth-20 methacrylate copolymer, acrylates/diacetoneacrylamide copolymer, acrylates/dimethicone copolymer, acrylates/dimethylaminoethyl methacrylate copolymer, acrylates/ethylhexyl acrylate copolymer, acrylates/ethylhexylacrylamide copolymer, acrylates/PVP copolymer, acrylates/steareth-20 itaconate copolymer, acrylates/steareth-20 methacrylate copolymer, acrylates/steareth-50 acrylate copolymer, acrylates/VA copolymer, acrylic acid/acrylonitrogens copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, adipic acid/neopentyl glycol/trimellitic anhydride copolymer, allyl stearate/VA copolymer, aminoethylacrylate phosphate/acrylates copolymer, ammonium acrylates copolymer, ammonium acrylates/acrylonitrogens copolymer, ammonium styrene/acrylates copolymer, ammonium VA/acrylates copolymer, amp-acrylates copolymer, amp-acrylates/diacetoneacrylamide copolymer, amp-acrylates/dimethylaminoethyl-methacrylate copolymer, ampd-acrylates/diacetoneacrylamide copolymer, α-olefin/MA copolymer, benzoic acid/phthalic anhydride/pentaerythritol/neopentyl glycol/palmitic acid copolymer, butadiene/acrylonitrile copolymer, butyl acrylate/hydroxyethyl methacrylate copolymer, butyl acrylate/styrene copolymer, butyl benzoic acid/phthalic anhydride/trimethylolethane copolymer, butyl ester of ethylene/MA copolymer, butyl ester of PVM/MA copolymer, butylene/ethylene copolymer, calcium/sodium PVM/MA copolymer, corn starch/acrylamide/sodium acrylate copolymer, dea-styrene/acrylates/DVB copolymer, decene/butene copolymer, dicyclopentadiene/tbutylcresol copolymer, diethylene glycolamine/epichlorohydrin/piperazine copolymer, diglycol/chdm/isophthalates/SIP copolymer, diglycol/isophthalates/SIP copolymer, dihydroxyethyl tallowamine/IPDI copolymer, dilinoleic acid/ethylenediamine copolymer, dilinoleyl alcohol/IPDI copolymer, dimethicone/mercaptopropyl methicone copolymer, dimethicone/sodium PG-propyl dimethicone thiosulfate copolymer, dimethiconol/IPDI copolymer, dimethiconol/silsesquioxane copolymer, dimethiconol/stearyl methicone/phenyl trimethicone copolymer, dimethylol urea/phenol/sodium phenol-sulfonate copolymer, dioctyldodecyl stearoyl dimer dilinoleate, DMAPA acrylates/acrylic acid/acrylonitrogens copolymer, dodecanedioic acid/cetearyl alcohol/glycol copolymer, ethyl ester of PVM/MA copolymer, ethylene/acrylic acid copolymer, ethylene/acrylic acid/VA copolymer, ethylene/calcium acrylate copolymer, ethylene/MA copolymer, ethylene/magnesium acrylate copolymer, ethylene/propylene copolymer, ethylene/sodium acrylate copolymer, ethylene/VA copolymer, ethylene/zinc acrylate copolymer, glycereth-7 hydroxystearate/IPDI copolymer, glycereth-7/IPDI copolymer, hydrogenated butylene/ethylene/styrene copolymer, hydrogenated ethylene/propylene/styrene copolymer, hydrogenated styrene/butadiene copolymer, hydrogenated styrene/methyl styrene/indene copolymer, hydrolyzed wheat protein/dimethicone copolyol phosphate copolymer, hydrolyzed wheat protein/PEG-20 acetate copolymer, isobutylene/isoprene copolymer, isobutylene/MA copolymer, isobutylene/sodium maleate copolymer, isoprene/pentadiene copolymer, isopropyl ester of PVM/MA copolymer, lauryl acrylate/VA copolymer, lauryl methacrylate/glycol dimethacrylate copolymer, methacryloyl ethyl betaine/acrylates copolymer, methoxy PEG-17/dodecyl glycol copolymer, methoxy PEG-22/dodecyl glycol copolymer, methylstyrene/vinyltoluene copolymer, nylon-12/6/66 copolymer, octadecene/MA copolymer, octylacrylamide/acrylates/butylaminoethyl methacrylate copolymer, PEG/PPG-125/30 copolymer, PEG/PPG-150/30 copolymer, PEG/PPG-17/6 copolymer, PEG/PPG-18/4 copolymer, PEG/PPG-23/50 copolymer, PEG/PPG-296/57 copolymer, PEG/PPG-300/55 copolymer, PEG/PPG-35/9 copolymer, PEG-100/IPDI copolymer, PEG-22/dodecyl glycol copolymer, PEG-45/dodecyl glycol copolymer, PEG-8/SMDI copolymer, phthalic anhydride/adipic acid/castor oil/neopentyl glycol/PEG-3/trimethylolpropane copolymer, phthalic anhydride/benzoic acid/trimethylolpropane copolymer, phthalic anhydride/butyl benzoic acid/propylene glycol copolymer, phthalic anhydride/glycerine/glycidyl decanoate copolymer, phthalic anhydride/trimellitic anhydride/glycols copolymer, piperylene/butane/pentene copolymer, polydiethyleneglycol adipate/IPDI copolymer, polyoxyisobutylene/methylene urea copolymer, PPG-12/SMDI copolymer, PPG-26/TDI copolymer, PPG-51/SMDI copolymer, PPG-7/succinic acid copolymer, PVM/MA copolymer, PVM/MA decadiene copolymer, PVP/acrylates/lauryl methacrylate copolymer, PVP/decene copolymer, PVP/dimethylaminoethylmethacrylate copolymer, PVP/DMAPA acrylates copolymer, PVP/eicosene copolymer, PVP/hexadecene copolymer, PVP/MA copolymer, PVP/VA copolymer, PVP/VA/itaconic acid copolymer, PVP/VA/vinyl propionate copolymer, PVP/vinyl caprolactam/DMAPA acrylates, ricinoleic acid/adipic acid/AEEA copolymer, sodium acrylate/vinyl alcohol copolymer, sodium acrylates copolymer, sodium acrylates/acrolein copolymer, sodium C4-12 olefin/maleic acid copolymer, sodium DVB/acrylates copolymer, sodium isooctylene/MA copolymer, sodium MA/diisobutylene copolymer, sodium methacrylate/styrene copolymer, sodium styrene/acrylamide copolymer, sodium styrene/acrylates copolymer, sodium styrene/acrylates PEG-10 dimaleate copolymer, sodium styrene/acrylates/divinyl-benzene copolymer, sodium styrene/PEG-10 maleate/nonoxynol-10 maleate/acrylates copolymer, sodium tauride acrylates/acrylic acid/acrylonitrogens copolymer, starch/acrylates/acrylamide copolymer, steareth-10 allyl ether/acrylates copolymer, stearoxymethicone/dimethicone copolymer, stearyl/aminopropyl methicone copolymer, stearylvinyl ether/MA copolymer, styrene/acrylamide copolymer, styrene/acrylates copolymer, styrene/acrylates/acrylonitrile copolymer, styrene/acrylates/ammonium methacrylate copolymer, styrene/allyl benzoate copolymer, styrene/butadiene copolymer, styrene/DVB copolymer, styrene/isoprene copolymer, styrene/MA copolymer, styrene/methylstyrene/indene copolymer, styrene/PVP copolymer, styrene/VA copolymer, sucrose benzoate/sucrose acetate iso-butyrate copolymer, sucrose benzoate/sucrose acetate iso-butyrate/butyl benzyl phthalate/methylmethacrylate copolymer, sucrose benzoate/sucrose acetate iso-butyrate/butyl benzyl phthalate copolymer, tea-acrylates/acrylonitrogens, terephthalic acid/isophthalic acid/sodium isophthalic acid sulfonate/glycol copolymer, trimethylpentanediol/isophthalic acid/trimellitic anhydride copolymer, tromethamine acrylates/acrylonitrogens copolymer, VA/butyl maleate/isobornyl acrylate copolymer, VA/crotonates, VA/crotonates/methacryloxybenzophenone-1 copolymer, VA/crotonates/vinyl neodecanoate copolymer, VA/crotonates/vinyl propionate copolymer, VA/crotonic acid/PEG-20M copolymer, VA/DBM copolymer, VA/isobutyl maleate/vinyl neodecanoate copolymer, VA/vinyl butyl benzoate/crotonates copolymer, and vinyl caprolactam/PVP/dimethyl-aminoethyl methacrylate copolymer.

Exemplary film-formers useful in the invention include, without limitation: adipic acid/trimellitic anhydride/neopentylglycol polyesters, adipic acid/isophthalic acid/neopentyl glycol/trimethylolpropane polyesters, phthalic anhydride/trimellitic anhydride/glycol polyesters, and the like.

Film formers expected to provide similar characteristics include, without limitation, VA/crotonic acid/PEG-20M copolymer, VA/crotonates/methacryloxybenzophenone-1 copolymer, stearoxymethicone/dimethicone copolymer, sodium styrene/acrylates PEG-10 dimaleate copolymer, sodium styrene/acrylates/divinyl-benzene copolymer, sodium styrene/PEG-10 maleate/nonoxynol-10 maleate/acrylates copolymer, polyoxyisobutylene/methylene urea copolymer, PPG-12/SMDI copolymer, PPG-26/TDI copolymer, PPG-51/SMDI copolymer, PPG-7/succinic acid copolymer, PEG/PPG-125/30 copolymer, PEG/PPG-150/30 copolymer, PEG/PPG-17/6 copolymer, PEG/PPG-18/4 copolymer, PEG/PPG-23/50 copolymer, PEG/PPG-296/57 copolymer, PEG/PPG-300/55 copolymer, PEG/PPG-35/9 copolymer, PEG-100/IPDI copolymer, PEG-22/dodecyl glycol copolymer, PEG-45/dodecyl glycol copolymer, PEG-8/SMDI copolymer, methoxy PEG-17/dodecyl glycol copolymer, methoxy PEG-22/dodecyl glycol copolymer, acrylamides/acrylates/DMAPA/methoxy PEG methacrylate copolymer, acrylates copolymer, acrylates/acetoacetoxyethyl methacrylate copolymer, adipic acid/dimethylaminohydroxypropyl diethylenetriamine copolymer, adipic acid/epoxypropyl diethylenetriamine copolymer, butyl acrylate/hydroxyethyl methacrylate copolymer, dihydroxyethyl tallowamine/IPDI copolymer, glycereth-7/IPDI copolymer, hydrolyzed wheat protein/PEG-20 acetate copolymer, methoxy PEG-17/dodecyl glycol copolymer, and methoxy PEG-22/dodecyl glycol copolymer.

Additional film formers may be from a class of silicone polymers known as siloxanes. In particular, polydimethylsiloxane (PDMS), more commonly known as dimethicone, is widely utilized in cosmetics and personal care. PDMS is found in a variety of rinse-off and leave-on products such as shampoos, hair conditioners, skin moisturizers and color cosmetics. Dimethicone exhibits unique physical properties that render it effective for overcoming the surface tension problems encountered with curable films, such as a gel polish nail enhancement.

Dimethicone is a hybrid inorganic-organic homopolymer comprised of dimethylsiloxane repeat units, i.e., the polymer consists of an inorganic siloxane backbone (—Si—O—) that bears two methyl (—CH₃) groups on each silicon (Si) atom. The resulting materials are hydrophobic liquids at room temperature with very low glass transition temperatures (Tg) and high permeability to gas, including water vapor.

Silicone polymers also have low surface tension, which is of primary importance to their spreading properties and film-forming ability. The critical surface tension of PDMS is 24 mN/m, which is greater than the liquid surface tension of 20.4 mN/m at 20° C. In comparison, the critical surface tension of mineral oil and deionized water are 30.4 mN/m at 25° C. and 72.0 mN/m, respectively. As a result, PDMS not only spreads easily but can wet almost all surfaces, including the natural nail.

A variety of silicone families with potential film-forming applications are based on the PDMS structure, including but not limited to: silicone fluids (from 0.65 mPa·s to a few thousand mPa·s viscosity), medium to high molecular weight PDMS and its emulsions (from 12,500 mPa·s fluid to silicone gums), silicone compounds (silica or silicate resin in silicone fluids), dimethicone crosspolymer (swollen and partially cross-linked elastomers), organofunctional silicones (alkylmethylsiloxane waxes, silicone polyethers), pressure sensitive silicone adhesives (PSAs), in-situ cured elastomer films, and silicone-organic combinations.

PDMS forms thin films on many organic substrates and is able to spread over its own absorbed film. The film-forming abilities of a particular silicone material are dependent on the molecular weight, structure and functionality of the polymer. Substantivity and durability of the film vary with the properties of the siloxane and the formulation, with hydrophobic films being water repellent, and as a result, more resistant to wash-off. These characteristics of easy spreading translate to an ability to aid spreading of other formulation ingredients, such as those in skin creams, lotions or topical drug formulations. In addition to their film-forming properties, some silicone materials also show an ability to gently adhere to proteinaceous substrates, such as the natural nail. The film-forming properties of linear PDMS can be adapted to meet specific needs. By adding a number of organoreactive groups to a polysiloxane, it is possible to create a silicon-based organoreactive molecule with the chemical characteristics of a carbon-based reactive molecule and the physical properties of a polysiloxane. The properties of basic PDMS materials can be changed by replacing some methyl groups with other organic groups or atoms (e.g., hydrogen, hydroxyl, vinyl, polyethylene oxide, alkoxy, phenyl, amine, or fluoro alkyl groups). Substitutions of this type can be useful when specific chemical and physical properties are desired, such as adhesion to a certain substrate, higher or lower polarity, better thermostability, enhanced hydrophilicity, compatibility with other organic materials or targeted reactivity. For example, the addition of tri- and tetrafunctional Si—O structures can be used to form silicone resins for improved substantivity and to modify feel. The addition of ethylene oxide or propylene oxide polymer chains to a siloxane (to provide a siloxane polyether) can improve compatibility between polar and non-polar materials. This capability to functionalize PDMS is critical for transforming liquid or highly flowable materials into thermoplastic or thermoset materials, such as are embodied by curable nail enhancements.

This organoreactive silicon-based molecule can be made to any size. Any number of reactive groups can be designed into it. Organoreactive groups can be attached to polysiloxanes in a number of ways, including: pendant to the polysiloxane backbone, forming “rake” or “comb” structures; at the ends of the polysiloxane chain, forming an ABA structure; or, at one end of the polysiloxane chain, forming an AB structure.

Examples of suitable siloxane film-formers include, without exception: Acrylate/Dimethicone Copolymer, Aminopropyl Dimethicone, Amodimethicone, Behenyl Dimethicone, Behenyl Dimethicone/Bis Vinyldimethicone Cross Polymer, Bis-PEG-12 Dimethicone, Bis-PEG-15 Methyl Ether Dimethicone, Bis-Stearoxydimethylsilane, Cerotyl Dimethicone, Cetyl Dimethicone, Cetyl Dimethicone/Bis Vinyldimethicone Cross Polymer, Cetyl Hexyl Dimethicone, Cetyl PEG/PPG-10/1 Dimethicone, Crotonic Acid/Vinyl C8-12 Isoalkyl Esters/VA/Bis-Vinyldimethicone Crosspolymer, Cyclomethicone, Cyclopentasiloxane, Dilinoleamidopropyl Dimethylamine Dimethicone PEG-10 Phosphate, Dilinoleamidopropyl Dimethylamine Dimethicone PEG-7 Phosphate, Dimethicone, Dimethicone PEG/PPG-7/4 Phosphate, Dimethicone PEG-10/15 Crosspolymer, Dimethicone PEG-20 Phosphate, Dimethicone PEG-7, Dimethicone PEG-7 Acetate, Dimethicone PEG-7 Isostearate, Dimethicone PEG-7 Lactate, Dimethicone PEG-7 Panthenyl Polyethoxy Phosphate, Dimethicone PEG-7 Undecylenate, Dimethicone PEG-8 Succinate, Dimethicone/Phenyl Vinyl Dimethicone Crosspolymer, Dimethicone/Vinyl Dimethicone Crosspolymer, Dimethicone/Vinyltrimethylsiloxysilicate Crosspolymer, Dimethiconol Behenate, Diphenyl Dimethicone, Disiloxane, Ethyl Methicone, Hexamethyldisiloxane, Hydroxypropyl Dimethicone Behenate, Isopropyl Phenyl Dimethicone, Lauryl PEG-8 Dimethicone, Lauryl PEG-9 Polydimethylsiloxyethyl Dimethicone, Lauryl Phenylisopropyl Dimethicone, Methyl Trimethicone, PEG/PPG-18/18 Dimethicone, PEG/PPG-20/20 Dimethicone, PEG/PPG-22/24 Dimethicone, PEG/PPG-25/25 Dimethicone, PEG-10 Dimethicone, PEG-11 Methyl Ether Dimethicone, PEG-12 Dimethicone, PEG-14 Dimethicone, PEG-17 Dimethicone, PEG-3 Dimethicone, PEG-6 Dimethicone, PEG-7 Dimethicone, PEG-8 Dimethicone, PEG-8 Distermonium Chloride, PEG-8 Trisiloxane, PEG-9 Dimethicone, Perfluorononyl Dimethicone, Perfluorononylethyl Carboxydecyl Dimethicone PEG-7 Phosphate, Perfluorononylethyl Carboxydecyl Lauryl/Behenyl Dimethicone, Perfluorononylethyl Carboxydecyl Lauryl Dimethicone, Perfluorononylethyl Carboxydecyl PEG-10 Dimethicone, Perfluorononylethyl Stearyl Dimethicone, PG-Dimethicone, Phenyl Isopropyl Dimethicone, Phenyltrimethicone Pheynyl Dimethicone, Polydecene & Cetyldimethicone/Bis Vinyldimethicone Crosspolymer, Polyphenylsilsesquioxane, Potassium Dimethicone PEG-7 Panthenyl Phosphate, Potassium Dimethicone PEG-7 Phosphate, Propoxytetramethyl Piperidinyl Dimethicone, Silicone Quaternium-20, Silicone Quaternium-24, Silicone Quaternium-8, Steardimonium Hydroxypropyl Panthenyl PEG-7 Dimethicone Phosphate Chloride, Stearyl Dimethicone, Trimethylsiloxyphenyl Dimethicone, Trimethylsilyamodimethicone, Trisiloxane, Vinyldimethyl/Trimethylsiloxysilicate Stearyl Dimethicone Crosspolymer, Dimethicone/Mercaptopropyl Methicone Copolymer, C20-24 Alkyl Dimethicone, C-24-28 Alkyl Dimethicone, C26-28 Alkyl Dimethicone, C30-45 Alkyl Dimethicone, C30-45 Alkyl Dimethicone/Polycyclohexene Oxide Crosspolymer, C30-45 Cetearyl Dimethicone Crosspolymer, C30-60 Alkyl Dimethicone, C32 Alkyl Dimethicone, C4-24 Alkyl Dimethicone/Divinyl-Dimethicone Crosspolymer, C6-8 Alkyl C3-6 Alkyl Glucoside Dimethicone, Dimethicone/Bis-isobutyl PPG-20 Crosspolymer, Dimethicone/Bis-Vinyldimethicone/Silsesquioxane Crosspolymer, Dimethicone/Divinyldimethicone/Silsesquioxane Crosspolymer, Dimethicone/Lauryl Dimethicone/Bis-Vinyldimethicone, Dimethicone/PEG-10/15 Crosspolymer, Dimethicone/PEG-10 Crosspolymer, Dimethicone/PEG-15 Crosspolymer, Dimethicone/Phenyl Vinyl Dimethicone Crosspolymer, Dimethicone/Polyglycerin-3 Crosspolymer, Dimethicone/PPG-20 Crosspolymer, Dimethicone/Silsesquioxane Copolymer, Dimethicone/Titanate Crosspolymer, Dimethicone/Vinyl Dimethicone Crosspolymer, Dimethicone/Vinyltrimethylsiloxysilicate Crosspolymer, Dimethicone Crosspolymer, Dimethicone Crosspolymer-3, Dimethicone Ethoxy Glucoside, Dimethicone Hydroxypropyl Trimonim Chloride, Dimethicone PEG/PPG-12/4 Phosphate, Dimethicone PEG/PPG-20/23 Benzoate, Dimethicone PEG/PPG-7/4 Phosphate, Dimethicone PEG-10 Phosphate, Dimethicone PEG-15 Acetate, Dimethicone PEG-7 Avocadoate, Dimethicone PEG-7 Cocoate, Dimethicone PEG-7 Isostearate, Dimethicone PEG-7 Lactate, Dimethicone PEG-7 Octyldodecyl Citrate, Dimethicone PEG-7 Olivate, Dimethicone PEG-7 Phosphate, Dimethicone PEG-7 Phthalate, Dimethicone PEG-7 Succinate, Dimethicone PEG-7 Sulfate, Dimethicone PEG-7 Undecylenate, Dimethicone PEG-8 Adipate, Dimethicone PEG-8 Beeswax, Dimethicone PEG-8 Borageate, Dimethicone PEG-8 Lanolate, Dimethicone PEG-8 Laurate, Dimethicone PEG-8 Meadowfoamate, Dimethicone PEG-8 Olivate, Dimethicone PEG-8 Phosphate, Dimethicone PEG-8 Phthalate, Dimethicone PEG-8 Polyacrylate, Dimethicone PEG-8 Succinate, Dimethicone PEG-Benzoate, Dimethicone Propyl PG-Betaine, Dimethicone Propylethylene-Diamine Behenate, Dimethicone Silylate, Dimethionce PG-Diethylmonium Chloride, Dimthicone PEG-8 Avocadoate, PEG/PEG-3/10 Dimethicone, PEG/PEG-4/12 Dimethicone, PEG/PPG-10/3 Oleyl Ether Dimethicone, PEG/PPG-12/16 Dimethicone, PEG/PPG-12/18 Dimethicone, PEG/PPG-14/4 Dimethicone, PEG/PPG-15/15 Dimethicone, PEG/PPG-15/5 Dimethicone, PEG/PPG-16/2 Dimethicone, PEG/PPG-16/8 Dimethicone, PEG/PPG-17/18 Dimethicone, PEG/PPG-18/12 Dimethicone, PEG/PPG-18/18 Dimethicone, PEG/PPG-18/6 Dimethicone, PEG/PPG-19/19 Dimethicone, PEG/PPG-20/15 Dimethicone, PEG/PPG-20/22 Methyl Ethyer Dimethicone, PEG/PPG-20/23 Dimethicone, PEG/PPG-20/29 Dimethicone, PEG/PPG-20/6 Dimethicone, PEG/PPG-22/23 Dimethicone, PEG/PPG-22/24 Dimethicone, PEG/PPG-23/6 Dimethicone, PEG/PPG-24/24 Methyl Ether Glycidoxy Dimethicone, PEG/PPG-25/25 Dimethicone, PEG/PPG-25/25 Dimethicone Acrylates Copolymer, PEG/PPG-27/27 Dimethicone, PEG/PPG-30/10 Dimethicone, PEG/PPG-6/11 Dimethicone, PEG/PPG-6/4 Dimethicone, PEG/PPG-8/14 Dimethicone, PEG/PPG-8/26 Dimethicone, PEG-10 Dimethicone/Vinyl Dimethicone Crosspolymer, PEG-10 Methyl Ether Dimethicone, PEG-11 Methyl Ether Dimethicone, PEG-12 Dimethicone Crosspolymer, PEG-12 Methyl Ether Lauroxy PEG-5 Amidopropyl Dimethicone, PEG-32 Methyl Ether Dimethicone, PEG-6 Methyl Ether Dimethicone, PEG-7 Methyl Ether Dimethicone, PEG-8 Dimethicone/Dimer Dilinoleic Acid Copolymer, PEG-8 Dimethicone/Polysorbate 20 Crosspolymer, PEG-8 Dimethicone Dimer Dilinoleate, PEG-8 Methyl Ether Dimethicone, PEG-8 PPG-8 Dimethicone, PEG-9 Methyl Ether Dimethicone, Triethoxysilylethyl Dimethicone/Methicone Copolymer, Triethoxysilylethyl Polydimethylsiloxyethyl Dimethicone, Triethoxysilylethyl Polydimethylsiloxyethyl Hexyl Dimethicone, and the like.

Exemplary siloxane film-formers useful in the invention include organofunctional silicones, such as silicones comprised of polyether segments such as polyoxyethylene (PEG) or polyoxypropylene (PPG) segments, regardless of number.

For example, the siloxane film-former may be a siloxane polyether containing at least one siloxane segment (such as a dimethicone segment) and at least one polyether segment. The polyether segment(s) may be, for example, polyoxyethylene segments, polyoxypropylene segments, or mixed polyoxyethylene/polyoxypropylene segments. The polyether segment(s) may be pendant to a siloxane backbone (to provide a rake-type structure) and/or arranged at one or both ends of a siloxane segment (to provide a block-type structure).

The amount of film-former in the composition is selected to be effective to provide desired properties. Typically, the composition contains at least 0.001 weight % film-former but no more than 20 weight % film-former. In various embodiments of the invention, the film-former content is at least 0.01, 0.1, 0.5, 1, 2 or 3 weight %. In other embodiments, the film-former content is no more than 15, 10 or 7 weight %. Generally speaking, when the film-former is a siloxane film-former, relatively low concentrations will be sufficient (e.g., 0.001 to 5 weight % or 0.01 to 1 weight %).

Suitable energy-curable resins for use in the present invention include any of the monomeric, oligomeric or polymeric compounds containing reactive carbon-carbon double bonds, particularly carbon-carbon double bonds that are reactive under free radical conditions. Such reactive monomer, oligomeric, or polymeric compounds may be (meth)acrylates, for example, i.e., compounds bearing one or more (meth)acrylate functional groups per molecule. The term (meth)acrylate, as will be appreciated by persons skilled in the art, encompasses both acrylates and methacrylates.

Typical examples of suitable energy-curable resins include esters and amides of acrylic and methacrylic acid. The esters of acrylic and methacrylic acid are herein termed (meth)acrylic esters. Specific, but not limiting, examples of mono-functional (meth)acrylic esters include: methyl(meth)acrylate, ethyl(meth)acrylate, hydroxypropyl(meth)acrylate, ethyl(meth)acrylate, butyl(meth)acrylate, hydroxyethyl(meth)acrylate, butoxyethyl(meth)acrylate, diethylaminoethyl(meth)acrylate, 2-ethylhexyl(meth)acrylate, ethoxyethyl(meth)acrylate, t-butyl aminoethyl(meth)acrylate, methoxyethylene glycol(meth)acrylate, phosphoethyl(meth)acrylate, silane functional (meth)acrylates, methoxy propyl(meth)acrylate, methoxy polyethylene glycol(meth)acrylate, phenoxyethylene glycol(meth)acrylate, phenoxypolyethylene glycol(meth)acrylate, 2-hydroxy-3-phenoxypropyl(meth)acrylate, phenoxy(meth)acrylate, phenoxy ethyl(meth)acrylate, 2-(meth)acryloxyethylsuccinic acid, 2-(meth)acryloylethylphthalic acid, 2-(meth)acryloyloxypropylphthalic acid, stearyl(meth)acrylate, isobornyl(meth)acrylate, 2-acetoacetoxyethyl(meth)acrylate, 3-chloro-2-hydroxypropyl(meth)acrylate, tetrahydrofurfuryl(meth)acrylate, trifluoroethyl(meth)acrylates, (meth)acrylamides and allyl monomers. Specific, but not limiting, examples of bifunctional (meth)acrylic esters include: 1,4-butanediol di(meth)acrylate, 1,6-hexanediol di(meth)acrylate, 1,9-nonanediol di(meth)acrylate, 1,10-decanediol di(meth)acrylate, 1,12-dodecanediol di(meth)acrylate, neopentyl glycol di(meth)acrylate, 2-methyl-1,8-octanediol di(meth)acrylate, glycerol di(meth)acrylate, ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, polypropylene glycol di(meth)acrylate, ethoxylated propylene glycol di(meth)acrylate, 1,3-butylene glycol di(meth)acrylate, ethoxylated polypropylene glycol di(meth)acrylate, polyethoxypropoxy di(meth)acrylate, ethoxylated bisphenol-A di(meth)acrylates, propoxylated bisphenol A di(meth)acrylates, propoxylated ethoxylated bisphenol-A di(meth)acrylates, glycidyl(meth)acrylate, bisphenol-A glycidyl(meth)acrylate, tricyclodecanedimethanol di(meth)acrylates, glycerin di(meth)acrylates, ethoxylated glycerin di(meth)acrylates, 2.2 bis[4-(acryloxy polyethoxy)phenyl]propane, bis acrylamides, 2-hydroxy-1-acryloxy-3-(meth)acryloxypropane, bis allyl ethers and allyl(meth)acrylates. Examples of tri-functional and higher (meth)acrylic esters include trimethylolpropane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol penta(meth)acrylate, dipentaerythritol hexa(meth)acrylate, and ethoxlated iscyanuric acid tri(meth)acrylates.

In one embodiment of the invention, the composition contains one or more urethane(meth)acrylates, which generally have one, two or more (meth)acrylate functional groups per molecule and at least one urethane group (linkage) per molecule. Examples of such substances include oligomeric urethanes based on polyester polyols and polyether polyols (e.g., polypropylene glycols, polytetramethylene glycols, polyethylene glycols, mixed polyethylene/polypropylene glycols, typically having number average molecular weights of from about 400 to about 3000) and aliphatic and aromatic diisocyanates capped with (meth)acrylate end-groups. Such substances may be described as urethane polyester (meth)acrylates and urethane polyether (meth)acrylates containing two or more (meth)acrylate groups per molecule. Urethane(meth)acrylates of this type may be prepared, for example, by reacting a polyether and/or polyester polyol with diisocyanate (aliphatic and/or aromatic) to form an isocyanate-capped prepolymer and then reacting the isocyanate groups of the prepolymer with a hydroxyalkyl(meth)acrylate to introduce the (meth)acrylate functional groups. Other suitable energy-curable resins include, but are not limited to, epoxy(meth)acrylates, epoxy urethane(meth)acrylates, (meth)acrylated polyester oligomers, (meth)acrylated acrylate oligomers, and the like. In various embodiments of the invention, the curable composition contains more urethane(meth)acrylate than any other single component of the composition. For example, the composition may comprise at least 20, 30, 40 or 50 weight % total of one or more urethane(meth)acrylates. The use of significant amounts of (meth)acrylate urethane oligomers has been found to improve the resistance of the cured composition to failure, typically embodied as edge chipping. Such oligomers also help to enhance film formation, durability, extended wear, shine, and soak-off. It is particularly preferred to use di(meth)acrylate urethane oligomers because such oligomers provide a strong, durable, flexible film, thereby avoiding the need to use a base coat and/or a top coat in addition to a coating of a composition in accordance with the invention. Particularly useful are urethane methacrylates such as EXOTHANE™ Elastomers, produced by Esstech, Inc. of Essington, Pa.

A composition in accordance with the invention may include one or more adhesion promoters, which may be considered to be substances which help to improve the adhesion of the cured composition to the nail. In one embodiment, the composition is formulated to contain sufficient adhesion promoter so as to avoid the need to pretreat the nail with acid, primer, base coat or the like before applying the composition in order to attain an adequate degree of adhesion between the cured composition and the nail surface. For example, the cured composition may be formulated such that it is capable of adhering to the nail under normal use conditions for at least a week or at least two weeks or even longer, while remaining capable of being removed from the nail within 10 minutes or within 5 minutes by soaking in acetone.

Suitable adhesion promoters for use in the invention include, but are not limited to, reactive (i.e., curable, polymerizable) compounds such as (meth)acrylates which also contain one or more other functional groups such as hydroxyl groups, carboxylic acid groups, phosphoric acid groups, ether groups, acetoxy groups and the like. Examples of adhesion promoters include photocurable resins such as hydroxyalkyl(meth)acrylates (e.g., hydroxyethyl(meth)acrylate, hydroxypropyl(meth)acrylate), pyromellitic dianhydride di(meth)acrylate, pyromellitic di(meth)acrylate, pyromellitic dianhydride glyceryl dimethacrylate (PMGDM), methacroyloxyethyl maleate, 2-hydroxyethyl(meth)acrylate, monoalkyl maleates, phthalic acid monoalkyl(meth)acrylates, phosphoric and carboxylic functional (meth)acrylates such as hydroxyethyl(meth)acrylate phosphate, maleate or succinate, hydroxypropyl(meth)acrylate phosphate, maleate or succinate, tetrahydrofurfuryl(meth)acrylate, glycerol phosphate di(meth)acrylate, ethoxyethyl(meth)acrylate, 2-phenoxyethyl methacrylate, alkoxylated bisphenol A di(meth)acrylate, alkoxylated trimethlolpropane triacrylate, alkoxylated nonyl phenol(meth)acrylate, methoxy or propoxy polyethylene glycol mono(meth)acrylate, alkoxylated pentaerythritol tri or tetraacrylate, diethylene glycol methyl ether(meth)acrylate, triethylene glycol ethyl ether(meth)acrylate, polyether acrylate oligomer, polypropylene glycol di(meth)acrylate, polyethylene glycol di(meth)acrylate, neopentylglycol di(meth)acrylate, trimethylolpropane triglycidyl ether, lauryl glycidyl ether, n-butyl glycidyl ether, 1,4-butanediol diglycidyl ether, poly(ethylene glycol) methyl ether(meth)acrylate, 1,6-hexanediol diglycidyl ether, trimethylolpropane triacrylate, HEMA polyethoxy ethyl methacrylate, polyethylene glycol methyl ether(meth)acrylate, aceto acetoxy ethyl methacrylate, and the like. Mixtures of different adhesion promoters may be employed. The amount of adhesion promoter in the curable composition may be varied as desired to achieve the needed adhesive strength in the composition when cured, but typically levels of at least 1, 2, 3, 4 or 5 weight % but no more than 30, 25 or 20 weight %, based on the total weight of the composition, will suffice. The use of an excessive amount of adhesion promoter may lead to difficulties in removing the cured composition from the nail surface when later desired. The adhesion promoter should also be selected to be one which does not adversely affect the gloss of the cured composition or create pull-back when the curable composition is applied to the nail. In particular advantageous embodiments of the invention, the curable composition is comprised of 0.1 to about 10 weight % or about 4 to about 5 weight % of pyromellitic dianhydride glyceryl dimethacrylate (PMGDM).

In certain embodiments, the curable compositions of the invention may contain one or more photoinitiators, at levels of 0.1 weight % or more for example. Photoinitiators are compounds which, when exposed to UV or visible light or other sources of radiant energy, help to initiate free radical polymerization or curing of the energy-curable resin or resins present in the compositions. Any of the photoinitiators known in the art may be employed, including, for example, benzoyldiphenylphosphinates, phenyl ketones, dimethyl ketals, benzophenone, benzophenone derivatives, benzyl ketones, monomeric hydroxyl ketones, polymeric hydroxyl ketones, alpha-aminoketones, acyl phosphine oxides, metallocenes, and the like and mixtures thereof. Generally speaking, the photoinitiator content of the compositions of the present invention may be up to 20% by weight.

If so desired, the inventive composition may be formulated to include one or more colorants such as pigments and/or dyes. Any of the colorants conventionally used in nail polishes and gels may be employed to impart a selected esthetic appearance to the coated nail. For example, the colorant may be in the form of a powdered pigment or a pigment dispersion, wherein a powdered pigment or mixture of powdered pigments is dissolved in a relatively non-volatile liquid medium such as castor oil, propylene glycol, or glycerin. The amount of colorant in the composition may vary, but concentrations up to 20% by weight are typically suitable. Since certain colorants may absorb the radiation used to cure the composition, the amount and/or type of photoinitiator may need to be adjusted to permit the composition to be cured within the desired period of exposure to the radiation.

The components of the curable composition are selected such that in its uncured state the composition has the consistency of a liquid or gel at room temperature and may be readily brushed, sprayed or otherwise applied onto a nail surface such that it forms a smooth layer of relatively uniform thickness. It is desirable that the composition have a viscosity at 25° C. of not less than 3,000 cPs and not more than 10,000 cPs. It is preferable that the composition have a viscosity at 25° C. of not less than 4,000 cPs and not more than 8,000 cPs. Mechanical shaping or other treatment of the cured composition is generally not needed in order to impart an esthetically pleasing appearance to the nail.

The composition may be applied directly to the natural nail surface, with no pretreatment of the nail surface or application of a base or primer coat. However, it may be helpful to clean the nail surface of any residual oils before applying the curable composition. This may be accomplished, for example, by wiping the nail surface with a suitable solvent capable of dissolving the oils. Once a layer of the composition has been applied (typically, over the entire surface of an individual nail), the layer is exposed to energy (e.g., actinic radiation such as UV or visible light) for a time effective to cause polymerization (curing) of the energy-curable components of the composition. The intensity and wavelength of the radiation are adjusted as needed and as will be readily appreciated by those skilled in the art in order to achieve the desired extent of curing, which will of course depend upon the structures and reactivities of the energy-curable components of the composition, the type and amount of photoinitiator selected, and other such factors.

Upon curing, the composition is thereby transformed from a liquid or gel to a solid, durable coating on the nail. One advantage of the present invention is that a top coat need not be further applied. That is, a cured composition in accordance with the invention is able to provide the esthetic characteristics generally desired in decorative nail enhancements (e.g., a smooth glossy finish that is resistant to everyday exposure to mechanical impact, abrasion, water immersion and the like for at least 10 or at least 14 days). However, such a top coat may be applied if so desired. In one embodiment of the invention, multiple coats of the cured composition are placed on a nail. For example, a first coat of a composition in accordance with the invention may be applied and cured to provide a first cured layer, with a second coat of the composition then being applied and cured. One or more additional coats may be similarly applied and cured, if desired. In this way, a nail enhancement of a desired thickness may be attained, which may help to improve the appearance and durability of the enhancement as compared to what can be achieved using a single coating or layer of the composition. After the final layer of the composition has been applied and cured, the outer surface of the cured composition may be wiped with alcohol or the like to remove the inhibition layer. The resulting coating will have a gloss equivalent to an industry-standard stand-alone top coat or nail lacquer.

Once cured, the compositions of the present invention may be conveniently removed from the nail surfaces to which they have been adhered by contacting the cured composition with an organic solvent such as acetone, butyl acetate, isopropyl alcohol, ethanol, ethyl acetate, methyl acetate, methyl ethyl ketone, and mixtures thereof. Such contacting may be carried out by soaking the coated nail in the organic solvent. Suitably, the composition is formulated such that it is capable of being completely removed from the nail surface after no more than 10, 9, 8, 7, 6, or 5 minutes of soaking in acetone at room temperature. In one advantageous embodiment, the cured composition is removable without abrading or other mechanical treatment.

### EXAMPLES

In one embodiment of the invention, the curable composition includes the following ingredients (the total of the listed components being 100 weight %):

| Description | Wt % |
|---|---|
| Polyester resin | 1-20 |
| Methacrylic Acid Tetrahydrofurfuryl Ester | 0-10 |
| 3,6,9-Trioxaundecamethylene dimethacrylate | 10-25 |
| Photoinitiator | 1-10 |
| Urethane dimethacrylate | 40-70 |
| 2,2-Bis(methacryloyloxymethyl)butyl methacrylate | 5-20 |

The above described system displays extraordinarily good application characteristics and very fast solvent removal after curing. However, there was poor durability and gloss was not at market standards.

In another embodiment of the invention, the curable composition corresponds to the following formulation (the total of the listed components being 100 weight %):

| Description | Wt % |
|---|---|
| Polyester resin | 15-30 |
| 3,6,9-Trioxaundecamethylene dimethacrylate | 0-15 |
| Photoinitiator | 1-10 |
| Urethane dimethacrylate | 40-65 |
| 2,2-Bis(methacryloyloxymethyl)butyl methacrylate | 5-20 |
| HEMA phosphate | 1-10 |

The above described system displayed wore well beyond 14 days. Yet, application pull-back was severe, the finish was matted and removal took 20 minutes.

In a preferred embodiment of the invention, the curable composition corresponds to the following formulation (the total of the listed components being 100 weight %):

| Description | Wt % |
|---|---|
| Vinyl acetate copolymer | 0.1-5 |
| Polyester resin | 1-15 |
| Methacrylic acid tetrahydrofurfuryl ester | 10-20 |
| 1,1,1-Trimethylolpropane triacrylate | 5-15 |
| Photoinitiator | 1-10 |
| Urethane dimethacrylate | 50-70 |
| Diethylene glycol monoethyl ether acrylate | 1-20 |
| Vinyl/acrylate polyester oligomer | 1-20 |

The above-described composition has polish-like application and viscosity. When applied to the nail there is no pull back from the side-wall and cuticle, and minimal pull back from the free-edge. The applied coating has a high gloss shine when cured in both LED and UV light. When soaked in acetone, the product completely removes from the nail after 5 minutes, with light scraping using a wood stick. There is minimal nail damage and no discoloration of the nail bed due to pigment bleeding. Adhesion of the coating to the nail may be further enhanced by the inclusion of 0.1 to 10 weight % of pyromellitic dianhydride glyceryl dimethacrylate (PMGDM) in the curable composition.

In another preferred embodiment of the invention, the curable composition corresponds to the following formulation (the total of the listed components being 100 weight %):

| Description | Wt % |
|---|---|
| Vinyl acetate copolymer | 0.1-5 |
| Polyester resin | 1-15 |
| Methacrylic acid tetrahydrofurfuryl ester | 10-20 |
| 1,1,1-Trimethylolpropane triacrylate | 5-15 |
| Photoinitiator | 1-10 |
| Urethane dimethacrylate | 40-60 |
| Poly(ethylene glycol) methyl ether (meth) acrylate | 2-12 |
| Vinyl/acrylate polyester oligomer | 1-10 |

The above-described composition has polish-like application and viscosity. When applied to the nail there is no pull back from the side-wall and cuticle, and minimal pull back from the free-edge. The applied coating has a high gloss shine when cured in both LED and UV light. Across a panel of testers, the coating proved to wear without defect for 14 days. When soaked in acetone, the product completely removes from the nail after 5 minutes, with light scraping using a wood stick. There is minimal nail damage and no discoloration of the nail bed due to pigment bleeding. Adhesion of the coating to the nail may be further enhanced by the inclusion of 0.1 to 10 weight % of pyromellitic dianhydride glyceryl dimethacrylate (PMGDM) in the curable composition.

The previous formulations demonstrate many desirable aspects, but all suffer from some amount of free-edge pull back, however slight. It is believed the leading boundary of the fluid holds an exceptionally high contact angle and the uncured gel pools to itself, which creates a minor aesthetic flaw on the open nail perimeter.

In an especially preferred embodiment of the invention, the curable composition corresponds to the following formulation (the total of the listed components being 100 weight %):

| Description | Wt % |
|---|---|
| Vinyl acetate copolymer | 0.1-5 |
| Polyester resin | 1-15 |
| Methacrylic acid tetrahydrofurfuryl ester | 10-20 |
| 1,1,1-Trimethylolpropane triacrylate | 5-15 |
| Photoinitiator | 1-10 |
| Urethane dimethacrylate | 50-70 |
| Diethylene glycol monoethyl ether acrylate | 1-20 |
| Vinyl/acrylate polyester oligomer | 1-20 |
| Siloxane polyether | 0.001-5 |

The above-described composition has extraordinary application characteristics. Reflecting the improved liquid/surface interface, when applied to the nail there is no pull back at any part of the coating circumference. The applied coating has a very high gloss shine when cured in both LED and UV light, equivalent to market-leading stand-alone top coats. When soaked in acetone, the product completely removes from the nail after 5 minutes, with light scraping using a wood stick. There is minimal nail damage and no discoloration of the nail bed due to pigment bleeding. Adhesion of the coating to the nail may be further enhanced by the inclusion of 0.1 to 10 weight % of pyromellitic dianhydride glyceryl dimethacrylate (PMGDM) in the curable composition.

In another especially preferred embodiment of the invention, the curable composition corresponds to the following formulation (the total of the listed components being 100 weight %):

| Description | Wt % |
|---|---|
| Polyester resin | 1-15 |
| Methacrylic acid tetrahydrofurfuryl ester | 10-20 |
| 1,1,1-Trimethylolpropane triacrylate | 5-15 |
| Photoinitiator | 1-10 |
| Urethane dimethacrylate | 50-70 |
| Diethylene glycol monoethyl ether acrylate | 1-20 |
| 3,3,5-Trimethylcyclohexyl acrylate | 0.1-5 |
| Siloxane polyether | 0.001-5 |
| Pyromellitic dianhydride glyceryl dimethacrylate | 0.1-10 |

The above-described composition has extraordinary application characteristics. Reflecting the improved liquid/surface interface, when applied to the nail there is no pull back at any part of the coating circumference. The applied coating has a very high gloss shine when cured in both LED and UV light, equivalent to market-leading stand-alone top coats. Using an industry standard 9 watt LED lamp, wear duration is greater than 7 days and may range up to 20 days, depending on the individual. When soaked in acetone, the product removes from the nail after 5 minutes, with light scraping using a wood stick. There is minimal nail damage and no discoloration of the nail bed due to pigment bleeding.

What is claimed is:

1. A composition useful as an energy-curable nail enhancement requiring no base coat or top coat when applied to a nail, wherein the composition comprises at least one film-former and at least one energy-curable resin and contains essentially no water or volatile non-reactive organic solvent.

2. The composition of claim 1, additionally comprising at least one colorant.

3. The composition of claim 1, additionally comprising at least one photoinitiator.

4. The composition of claim 1, additionally comprising at least one adhesion promoter.

5. The composition of claim 1, wherein the composition additionally comprises at least one adhesion promoter selected from the group consisting of (meth)acrylates containing at least one functional group selected from hydroxyl groups, carboxylic acid groups, phosphoric acid groups, ether groups and acetoxy groups per molecule.

6. The composition of claim 1, wherein the composition comprises at least one film-former which is a polyester.

7. The composition of claim 1, wherein the composition comprises at least one film-former which is a polyester obtained by condensation of at least one reactant selected from the group consisting of aromatic and aliphatic di- and tri-carboxylic acids and anhydrides thereof and at least one reactant selected from the group consisting of di- and tri-alcohols.

8. The composition of claim 1, wherein the composition comprises at least one film-former which is an adipic acid/trimellitic anhydride/neopentylglycol polyester.

9. The composition of claim 1, wherein the composition comprises at least 0.1 weight percent polyester film-former.

10. The composition of claim 1, wherein at least one film-former is soluble in an organic solvent.

11. The composition of claim 1, wherein the at least one energy-curable resin contains one or more (meth)acrylate functional groups.

12. The composition of claim 1, wherein the composition comprises at least one oligomeric energy-curable resin.

13. The composition of claim 1, wherein the composition comprises at least one energy-curable resin containing two or more (meth)acrylate functional groups.

14. The composition of claim 1, wherein the composition comprises at least one energy-curable resin containing three or more (meth)acrylate functional groups.

15. The composition of claim 1, wherein the composition comprises at least one urethane polyether(meth)acrylate or urethane polyester(meth)acrylate containing two or more (meth)acrylate groups per molecule.

16. The composition of claim 1, wherein the composition comprises at least one urethane(meth)acrylate.

17. The composition of claim 1, wherein the composition comprises at least one film-former which is a siloxane.

18. The composition of claim 1, wherein the composition comprises at least one film-former which is a dimethicone polyether containing at least one dimethicone segment and at least one polyether segment, the polyether segment being selected from the group consisting of polyoxyethylene segments, polyoxypropylene segments, and mixed polyoxyethylene/polyoxypropylene segments.

19. The composition of claim 1, wherein the composition comprises at least 0.001 weight percent siloxane film-former.

20. The composition of claim 1, wherein the composition comprises at least one siloxane film-former containing organofunctional groups soluble in an organic solvent.

21. A nail having an external surface upon which is directly applied a layer of a composition in accordance with claim 1.

22. The nail of claim 21, wherein the layer has been energy-cured.

23. The nail of claim 22, wherein the energy-cured layer is capable of being removed by soaking in acetone at room temperature within ten minutes.

24. The nail of claim 21, wherein no further layer is present on top of the layer of the composition.

25. A nail having an external surface upon which is directly applied a first energy-cured layer of a composition in accordance with claim 1 and, on top of the first energy-cured layer, a second energy-cured layer of a composition in accordance with claim 1.

26. The nail of claim 25, wherein no further layer is present on top of the second energy-cured layer.

27. A method of decorating a nail, comprising the steps of applying a layer of a composition in accordance with claim 1 directly to an external surface of the nail and energy-curing the composition.

28. The method of claim 27, comprising an additional step of applying a second layer of the composition to the top of the energy-cured composition and energy-curing the composition in the second layer.

29. A energy-cured composition obtained by energy-curing a composition in accordance with claim 1.

30. A composition useful as an energy-curable nail enhancement requiring no base coat or top coat when applied to a nail, wherein the composition contains essentially no water or volatile non-reactive organic solvent and is comprised of 0.1-5 weight % vinyl acetate copolymer, 1-15 weight % polyester resin, 10-20 weight % methacrylic acid tetrahydrofurfuryl ester, 5-15 weight % 1,1,1-trimethylolpropane triacrylate, 1-10 weight % photoinitiator, 50-70 weight % urethane dimethacrylate, 1-20 weight % diethylene glycol monoethyl ether acrylate, and 1-20 weight % vinyl/acrylate polyester oligomer.

31. A composition useful as an energy-curable nail enhancement requiring no base coat or top coat when applied to a nail, wherein the composition contains essentially no water or volatile non-reactive organic solvent and is comprised of 0.1-5 weight % vinyl acetate copolymer, 1-15 weight % polyester resin, 10-20 weight % methacrylic acid tetrahydrofurfuryl ester, 5-15 weight % 1,1,1-trimethylolpropane triacrylate, 1-10 weight % photoinitiator, 40-60 weight % urethane dimethacrylate, 2-12 weight % poly(ethylene glycol) methyl ether(meth)acrylate, and 1-10 weight % vinyl/acrylate polyester oligomer.

32. A composition useful as an energy-curable nail enhancement requiring no base coat or top coat when applied to a nail, wherein the composition contains essentially no water or volatile non-reactive organic solvent and is comprised of 0.1-5 weight % vinyl acetate copolymer, 1-15 weight % polyester resin, 10-20 weight % methacrylic acid tetrahydrofurfuryl ester, 5-15 weight % 1,1,1-trimethylolpropane triacrylate, 1-10 weight % photoinitiator, 50-70 weight % urethane dimethacrylate, 1-20 weight % diethylene glycol monoethyl ether acrylate, 1-20 weight % vinyl/acrylate polyester oligomer, and 0.001-5 weight % siloxane polyether.

33. A composition useful as an energy-curable nail enhancement requiring no base coat or top coat when applied to a nail, wherein the composition contains essentially no water or volatile non-reactive organic solvent and is comprised of 1-15 weight % polyester resin, 10-20 weight % methacrylic acid tetrahydrofurfuryl ester, 5-15 weight % 1,1,1-trimethyolpropane triacrylate, 1-10 weight % photoinitiator, 50-70 weight % urethane dimethacrylate, 1-20 weight % diethylene glycol monoethyl ether acrylate, 0.1-5 weight % 3,3,5-trimethylcyclohexyl acrylate, 0.001-5 weight % siloxane polyether, and 0.1-10 weight % pyromellitic dianhydride glyceryl dimethacrylate.
